(19) United States
(12) Patent Application Publication
(10) Pub. No.: US 2025/0073378 A1
(43) Pub. Date: Mar. 6, 2025

(54) WHITE BLOOD CELL COLLECTION APPARATUS AND WHITE BLOOD CELL COLLECTION METHOD

(71) Applicants: CANON KABUSHIKI KAISHA, Tokyo (JP); Canon Medical Systems Corporation, Otawara-shi (JP)

(72) Inventors: Akane KUBOYAMA, Kawasaki (JP); Tsutomu MIKI, Komae (JP); Yuhi HIGUCHI, Tokyo (JP)

(73) Assignees: CANON KABUSHIKI KAISHA, Tokyo (JP); Canon Medical Systems Corporation, Otawara-shi (JP)

(21) Appl. No.: 18/815,948

(22) Filed: Aug. 27, 2024

(30) Foreign Application Priority Data
Aug. 31, 2023 (JP) 2023-141413

(51) Int. Cl. A61M 1/02

(52) U.S. Cl. CPC A61M 1/0218; A61M 2202/0439

(57) ABSTRACT

According to one embodiment, a white blood cell collection apparatus includes a first container, a second container, a filter, a branching portion, and a controller. The controller controls liquid delivery operation through the branching portion. The liquid delivery operation includes: sending blood in the first container from the branching portion to the filter to cause the filter to capture white blood cells contained in the blood; pushing back the blood remaining in the branching portion toward the first container by sending the wash liquid in the second container from the branching portion toward the first container after the capturing; and washing the filter by sending the wash liquid in the second container from the branching portion to the filter after the pushing back.

### CROSS-REFERENCE TO RELATED APPLICATION

This application is based upon and claims the benefit of priority from prior Japanese Patent Application No. 2023-141413, filed Aug. 31, 2023, the entire contents of which are incorporated herein by reference.

### FIELD

Embodiments described herein relate generally to a white blood cell collection apparatus and a white blood cell collection method.

### BACKGROUND

A cell production process for producing cells includes, for example, a step of collecting cells from tissues of a donor and a step of culturing the collected cells. Here, the step of collecting cells is generally performed by a combination of a centrifuge and procedure by an engineer. For example, in a case where white blood cells are collected from blood of a donor, a liquid obtained by separating components of blood is obtained in a test tube by a centrifuge. The liquid in the test tube is separated into red blood cells (45%), white blood cells and platelets (<1%), and plasma (55%) in order from the bottom of the test tube. The engineer collects white blood cells manually using a pipette for each test tube. However, the step of collecting white blood cells by such manual work is not suitable for automation.

On the other hand, from the viewpoint of automation, a method is known in which a filter is provided in part of a pipe through which blood flows to collect white blood cells from blood flowing through the pipe. In this method, for example, white blood cells are captured by the filter from blood flowing in a certain direction, the filter is washed by a wash liquid flowing in the same direction, and the white blood cells are collected from the filter by a wash liquid flowing in the opposite direction.

### BRIEF DESCRIPTION OF DRAWINGS

FIG. 1 is a block diagram showing an example of a configuration of a white blood cell collection apparatus according to one embodiment.

FIG. 2 is a flowchart for explaining an example of operation in one embodiment.

FIG. 3 is a schematic diagram for explaining operation of step ST1 in FIG. 2.

FIG. 4 is a schematic diagram for explaining operation of step ST2 in FIG. 2.

FIG. 5 is a schematic diagram for explaining operation of step ST3 in FIG. 2.

FIG. 6 is a schematic diagram for explaining operation of step ST4 in FIG. 2.

FIG. 7 is a schematic diagram for explaining operation of step ST5 in FIG. 2.

FIG. 8 is a schematic diagram for explaining initial operation of step ST6 in FIG. 2.

FIG. 9 is a schematic diagram for explaining operation of step ST6 in FIG. 2.

FIG. 10 is a schematic diagram for explaining a problem of a comparative example with respect to one embodiment.

FIG. 11 is a schematic diagram for explaining an effect of one embodiment with respect to the comparative example.

FIG. 12 is a block diagram showing a configuration of a white blood cell collection apparatus according to a modification of one embodiment.

FIG. 13 is a schematic diagram for explaining a configuration of a collection cartridge in FIG. 12.

FIG. 14 is a schematic diagram showing part of a configuration in which the collection cartridge of FIG. 13 includes a diaphragm.

FIG. 15 is a schematic diagram showing part of a configuration in which the collection cartridge of FIG. 13 includes a bellows.

FIG. 16 is a block diagram showing a configuration of a white blood cell collection apparatus according to another modification of the embodiment.

FIG. 17 is a schematic diagram showing part of a configuration in which the collection cartridge of FIG. 16 includes a balloon.

FIG. 18 is a block diagram showing a configuration of a white blood cell collection apparatus according to still another modification of the embodiment.

### DETAILED DESCRIPTION

In general, according to one embodiment, a white blood cell collection apparatus includes a first container, a second container, a filter, a branching portion, and a controller. The first container stores blood. The second container stores a wash liquid. The blood sent from the first container and the wash liquid sent from the second container pass through the filter. The controller controls liquid delivery operation through the branching portion. The liquid delivery operation includes capturing, pushing back, and washing. The capturing includes capturing white blood cells contained in blood by the filter by sending the blood in the first container from the branching portion to the filter. The pushing back includes pushing back the blood remaining in the branching portion toward the first container by sending the wash liquid in the second container from the branching portion toward the first container after the capturing. The washing includes washing the filter by sending the wash liquid in the second container from the branching portion to the filter after the pushing back.

Hereinafter, a white blood cell collection apparatus and a white blood cell collection method according to one embodiment will be described with reference to the drawings. Note that in the following description, a case where the white blood cell collection apparatus collects white blood cells to be used for producing induced pluripotent stem (iPS) cells will be described as an example. However, the white blood cell collection apparatus and the white blood cell collection method are not limited to production of iPS cells, and can be used for any purpose of collecting white blood cells.

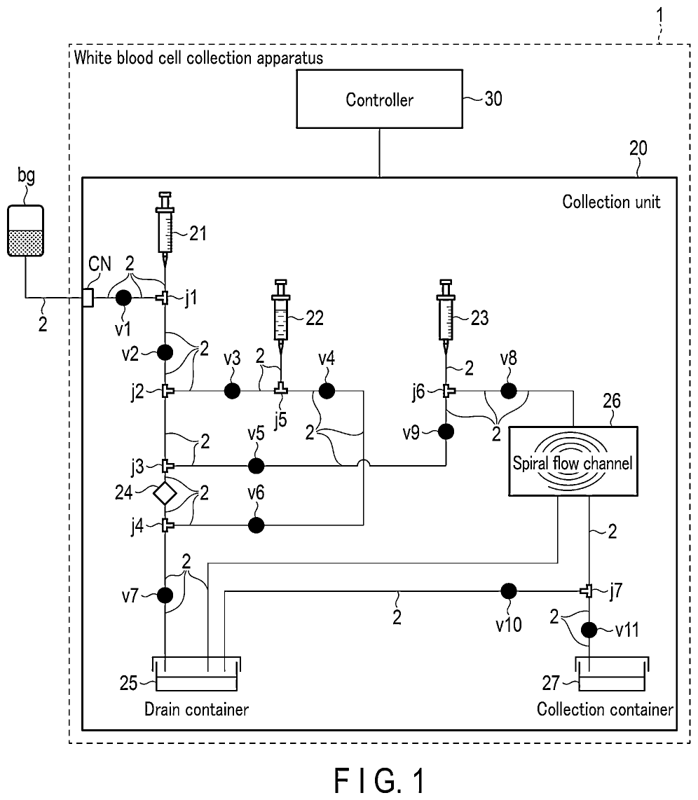

FIG. 1 is a block diagram showing an example of a configuration of the white blood cell collection apparatus according to one embodiment. A white blood cell collection apparatus 1 includes a collection unit 20 and a controller 30.

Here, the collection unit 20 includes a first syringe 21, a second syringe 22, a third syringe 23, a filter 24, a drain container 25, a spiral flow channel 26, a collection container 27, a plurality of pipes 2, joints j1 to j7, valves v1 to v11, and a connector CN. The pipe 2 of a blood bag bg is detachably connected to the connector CN from the outside. The joints j1 to j7 have a substantially T-shaped branched shape, are connected to the three pipes 2, and allow the three pipes 2 to communicate with each other. Note that, among the joints j1 to j7, the joint j2 is an example of a branching portion communicating with each of the first container (first syringe 21), the second container (second syringe 22), and the filter 24. In other words, the branching portion is the joint j2 that connects the pipe 2 of a flow channel communicating with the first syringe 21, the pipe 2 of a flow channel communicating with the second syringe 22, and the pipe 2 of a flow channel communicating with the filter 24. The pipes 2 are provided with the valves v1 to v11 as appropriate. The valves v1 to v11 elastically deform and close the elastically deformable pipes 2 to close the pipes 2 and release the pipes 2 to open the pipes 2. As such valves v1 to v11, for example, pinch valves whose opening and closing are controlled by the controller 30 can be used. Also, the pipes 2 are each constituted by, for example, an airtight flexible tubular member such as a vinyl or plastic tube. Each liquid flowing through the pipes 2 is thus inhibited or prevented from being exposed to the ambient air.

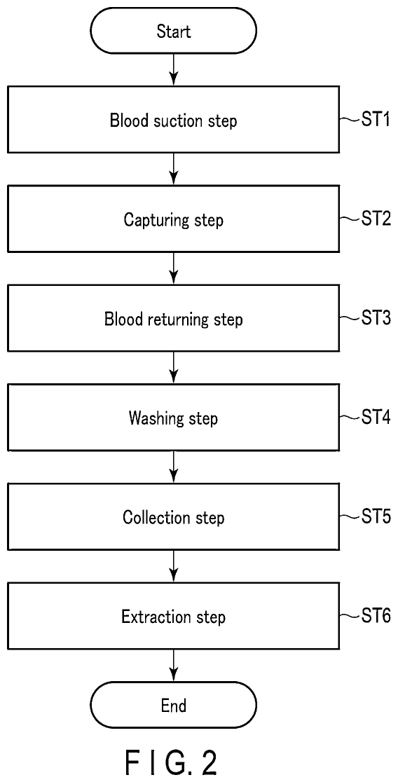

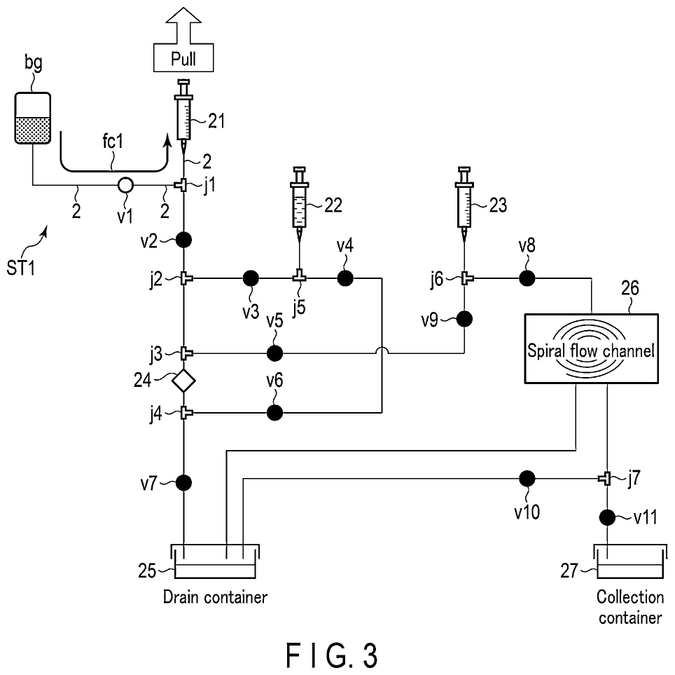

As will be described later with reference to FIGS. 2 to 9, such a collection unit 20 is formed such that various closed-system flow channels can be switched by open states of the valves v1 to v11. For example, as shown in FIGS. 2 and 3, a first flow channel fc1 from the blood bag bg to the first syringe 21 via the pipe 2, the valve v1, the joint j1, and the pipe 2 is formed by the open state of the valve v1. Note that in FIG. 3, a white circle of the valve v1 represents an open state, and a black circle of the valves v2 to v11 represents a closed state. Hereinafter, similarly, white circles of the valves v1 to v11 represent open states of the valves v1 to v11, and black circles of the valves v1 to v11 represent closed states of the valves v1 to v11.

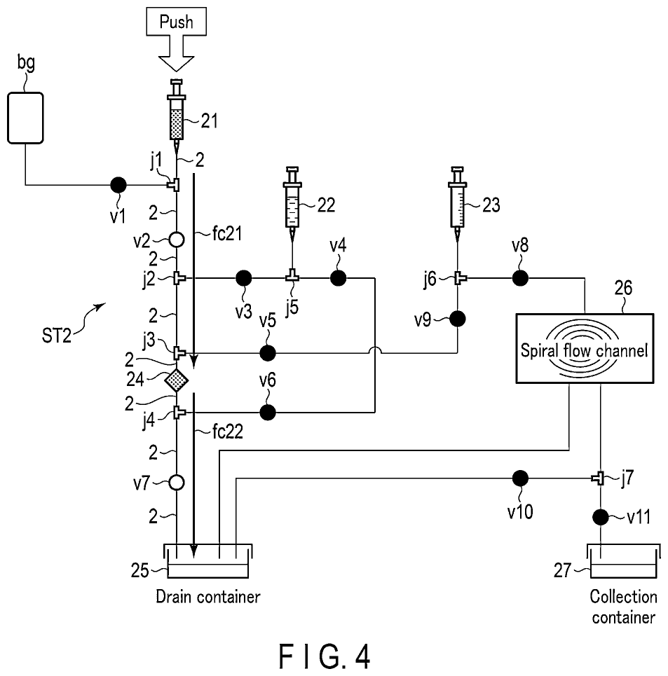

Further, as shown in FIG. 4, a 21st flow channel fc21 from the first syringe 21 to the filter 24 via the pipe 2, the joint j1, the pipe 2, the valve v2, the joint j2, the pipe 2, the joint j3, and the pipe 2 is formed by the open state of the valve v2. In addition, a 22nd flow channel fc22 from the filter 24 to the drain container 25 via the pipe 2, the joint j4, the pipe 2, and the valve v7 is formed by the open state of the valve v7. The 21st flow channel fc21 and the 22nd flow channel fc22 form a continuous second flow channel fc2.

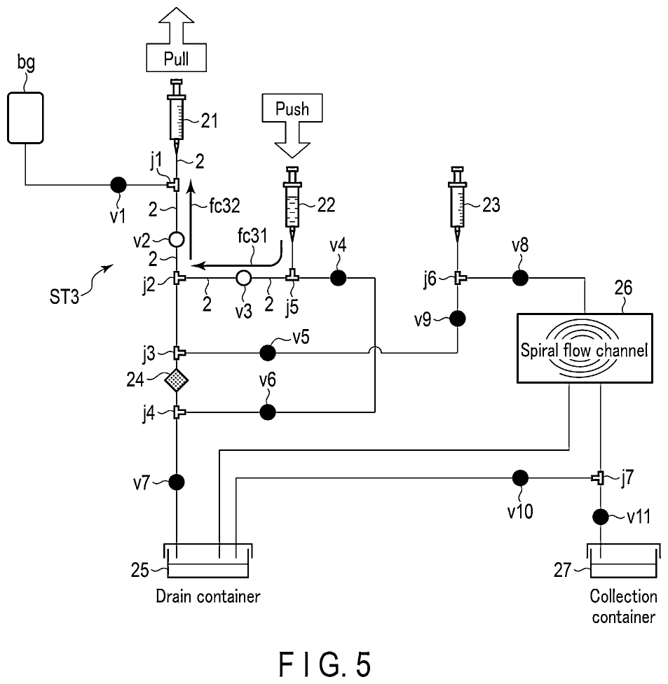

As shown in FIG. 5, a 31st flow channel fc31 from the second syringe 22 to the joint j2 via the pipe 2, the joint j5, the pipe 2, and the valve v3 is formed by the open state of the valve v3. In addition, a 32nd flow channel fc32 from the joint j2 to the first syringe 21 via the pipe 2, the valve v2, the joint j1, and the pipe 2 is formed by the open state of the valve v2. The 31st flow channel fc31 and the 32nd flow channel fc32 form a continuous third flow channel fc3.

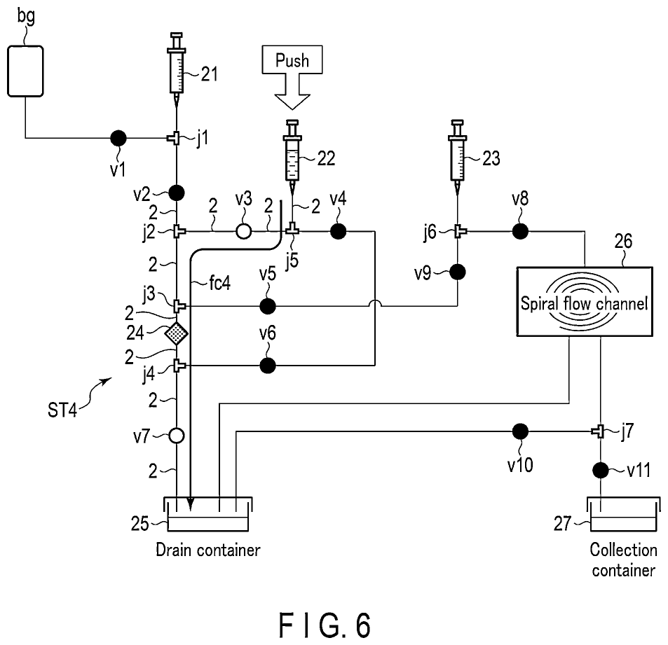

Further, as shown in FIG. 6, a fourth flow channel fc4 from the second syringe 22 to the drain container 25 via the pipe 2, the joint j5, the pipe 2, the valve v3, the joint j2, the pipe 2, the joint j3, the pipe 2, the filter 24, the pipe 2, the joint j4, the pipe 2, and the valve v7 is formed by the open states of the valves v3 and v7.

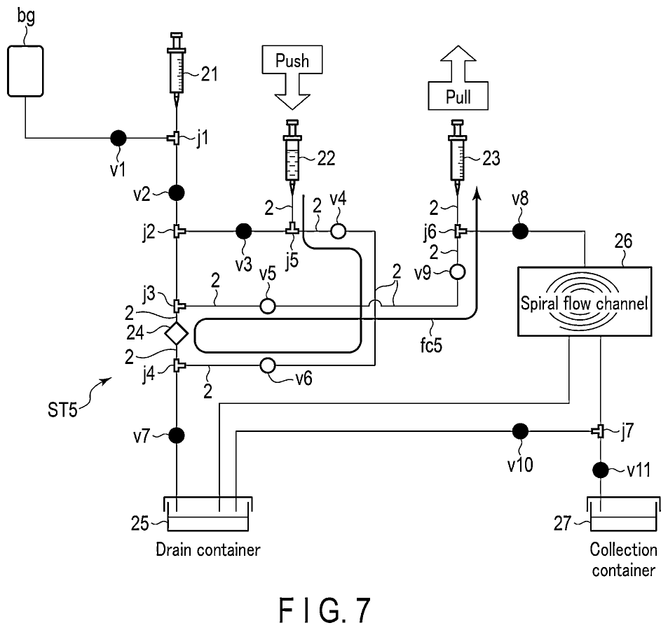

Further, as shown in FIG. 7, a fifth flow channel fc5 from the second syringe 22 to the third syringe 23 via the pipe 2, the joint j5, the pipe 2, the valves v4 and v6, the joint j4, the pipe 2, the filter 24, the pipe 2, the joint j3, the pipe 2, the valves v5 and v9, the joint j6, and the pipe 2 is formed by the open states of the valves v4 to v6 and v9.

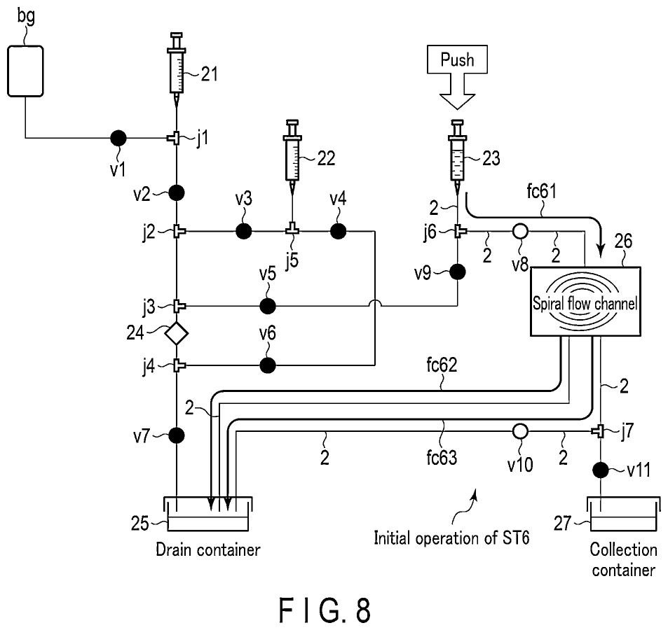

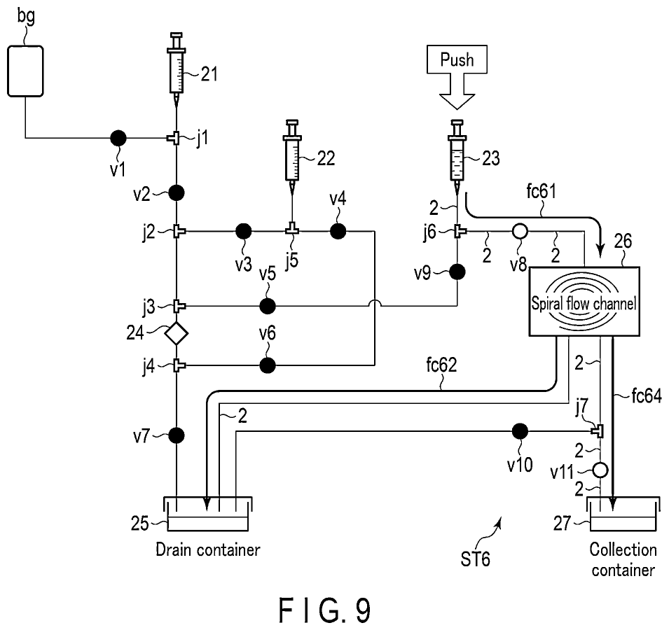

As shown in FIG. 8, a 61st flow channel fc61 from the third syringe 23 to the spiral flow channel 26 via the pipe 2, the joint j6, the pipe 2, and the valve v8 is formed by the open state of the valve v8. Further, a 62nd flow channel fc62 from the spiral flow channel 26 to the drain container 25 via the pipe 2 is always formed. Further, a 63rd flow channel fc63 from the spiral flow channel 26 to the drain container 25 via the pipe 2, the joint j7, the pipe 2 and the valve v10 is formed by the open state of the valve v10. As shown in FIG. 9, a 64th flow channel fc64 from the spiral flow channel 26 to the collection container 27 via the pipe 2, the joint j7, the pipe 2, and the valve v11 is formed by the open state of the valve v11.

The first syringe 21 includes an injector including a cylinder and a piston. The first syringe 21 can store a liquid such as blood in the cylinder and has sealability so as to suppress or prevent the liquid from being exposed to the outside air. A distal end portion of the first syringe 21 is connected to the joint j1 via the pipe 2. The first syringe 21 is controlled by the controller 30, discharges the blood in the cylinder from the distal end portion by the piston pushing action and sucks the blood in the blood bag bg or the blood in the pipe 2 from the distal end portion by the piston pulling action. Such a first syringe 21 is suitable for quantitative and accurate liquid delivery. The first syringe 21 is an example of a first container that stores blood.

The second syringe 22 includes an injector including a cylinder and a piston. The second syringe 22 can store a wash liquid in the cylinder and has sealability so as to suppress or prevent the wash liquid from being exposed to the outside air. The distal end portion of the second syringe 22 is connected to the joint j5 via the pipe 2. The second syringe 22 is controlled by the controller 30 and discharges the wash liquid in the cylinder from the distal end portion by the piston pushing action. The wash liquid is, for example, phosphate buffered saline (PBS), or the like, and a liquid having less influence of damage on white blood cells is used. Such a second syringe 22 is suitable for quantitative and accurate liquid delivery. The second syringe 22 is an example of a second container that stores the wash liquid.

The third syringe 23 includes an injector including a cylinder and a piston. The third syringe 23 can store the collected blood component and a liquid such as a wash liquid in the cylinder and has sealability so as to suppress or prevent the liquid from being exposed to the outside air. The distal end portion of the third syringe 23 is connected to the joint j6 via the pipe 2. The third syringe 23 is controlled by the controller 30, discharges the liquid in the cylinder from the distal end portion by the piston pushing action and sucks the liquid in the pipe 2 from the distal end portion by the piston pulling action. Such a third syringe 23 is suitable for quantitative and accurate liquid delivery. The third syringe 23 may be referred to as a third container.

The filter 24 is provided between the first syringe 21 and the drain container 25 and captures white blood cells contained in the blood sent from the first syringe 21. The filter 24 is constituted by a container including a filter body having an opening capable of capturing white blood cells and through which other substances (red blood cells, platelets) having a smaller particle size than the white blood cells can pass. That is, each white blood cell as a target cell having a particle size larger than the opening of such meshes is caught by the filter 24. Red blood cells, etc., not handled as target cells in this example, each have a particle size smaller than the mesh opening, and therefore, they pass through the filter 24. As the filter 24, for example, a white blood cell removing filter such as a trade name Acrodisc WBC syringe filter (AP-4951) manufactured by PALL Corporation or a trade name SEPACell RZ (RZ-1000N, RZ-2000N) manufactured by Asahi Kasei Medical Co., Ltd. can be appropriately used. The filter 24 is an example of a filter through which the blood sent from the first container and the wash liquid sent from the second container pass.

The drain container 25 is a container that stores a drained liquid. The drain container 25 stores, as the drained liquid, blood components and the wash liquid that have passed through the filter 24. The drain container 25 may be a container capable of storing the drained liquid, such as a flask or a bag.

The spiral flow channel 26 is a spiral flow channel that separates particles from a fluid according to a size of the particles contained in the fluid if the fluid containing white blood cells is sent. Specifically, the spiral flow channel 26 is configured to extract CD34 positive cells from white blood cells sent from the third syringe 23 and flowing through the spiral flow channel, and sends the extracted CD34 positive cells toward the collection container 27 and sends the remaining white blood cells toward the drain container 25. The spiral flow channel 26 may be called a “spiral inertial filtering device”, or a “liquid processing device”. Note that the techniques of a spiral inertial filtering device are available from, for example, the disclosures in Jpn. PCT National Publication No. 2015-532198. The spiral flow channel 26 is an example of a spiral flow channel through which white blood cells are to be sent.

The collection container 27 is a container that stores CD34 positive cells contained in the collected white blood cells. The collection container 27 stores the CD34 positive cells extracted by the spiral flow channel 26. The collection container 27 may be a container capable of culturing CD34 positive cells, such as a well plate, a flask, a dish, or a bag.

On the other hand, the controller 30 controls operation of the entire white blood cell collection apparatus 1. For example, the controller 30 switches and forms each of the first flow channel fc1 to the 64th flow channel fc64 by opening/closing control of the valves v1 to v11. In addition, the controller 30 controls discharge operation and suction operation to be performed by the first syringe 21, the second syringe 22, and the third syringe 23 with respect to the formed flow channel. Such operation of the entire white blood cell collection apparatus 1 includes liquid delivery operation through the joint j2 and collection operation different from the liquid delivery operation. The entire operation of the white blood cell collection apparatus 1 may be referred to as blood cell separation operation. The controller 30 is an example of a controller that controls the liquid delivery operation passing through the branching portion.

The liquid delivery operation includes the filter 24 capturing white blood cells contained in the blood by sending the blood in the first syringe 21 from the joint j2 to the filter 24, pushing back the blood remaining in the joint j2 toward the first syringe 21 by sending the wash liquid in the second syringe 22 from the joint j2 toward the first syringe 21 after the capturing, and washing the filter 24 by sending the wash liquid in the second syringe 22 from the joint j2 to the filter 24 after the pushing back.

To be more specific, in the liquid delivery operation, the valve v2 disposed between the joint j2 and the first syringe 21 is controlled to be open and closed by the controller 30. For example, the capturing includes controlling the valve v2 to be in an open state. The pushing back includes controlling the valve v2 to be in the open state and sending the wash liquid to at least a position closer to the first syringe 21 than the valve v2. The washing includes controlling the valve v2 to be in a closed state. The pushing back may include sending the wash liquid to the first syringe 21.

The collection operation is operation of collecting white blood cells from the filter 24 after the liquid delivery operation. In the collection operation, a direction of liquid delivery flowing through the filter 24 is opposite to a direction of liquid delivery flowing through the filter 24 in the liquid delivery operation. For example, in the liquid delivery operation, white blood cells are captured by the filter 24 from blood flowing in a certain direction, and the filter 24 is washed with the wash liquid flowing in the same direction. On the other hand, in the collection operation, the white blood cells are collected from the filter 24 by the wash liquid flowing in the direction opposite to the direction in the liquid delivery operation. Specifically, in the collection operation, the white blood cells are collected from the filter 24 by causing the wash liquid to flow in a direction from the filter 24 side toward the white blood cells side with respect to the filter 24 that has captured the white blood cells. In addition, the collection operation may include: collecting white blood cells from the filter 24 after washing the filter 24; and sending the collected white blood cells to the spiral flow channel 26 so that the spiral flow channel 26 extracts CD34 positive cells from the white blood cells. As the controller 30, for example, processing circuitry including a processor such as a CPU or an MPU and a memory such as a ROM or a RAM as hardware resources can be appropriately used.

An operation example of the white blood cell collection apparatus configured as described above will be described with reference to the flowchart of FIG. 2 and the schematic diagrams of FIGS. 3 to 9. In the following description, it is assumed that blood collected from a donor is stored in the blood bag bg, and physiological saline (PBS) is stored as the wash liquid in the second syringe 22. In addition, it is assumed that the first syringe 21 and the third syringe 23 are empty. In addition, it is assumed that all the valves v1 to v11 are in a closed state in which the pipe 2 is closed. In addition, steps ST1 to ST6 in the operation of the white blood cell collection apparatus 1 are executed under the control of the controller 30 without user intervention.

In a blood suction step of step ST1, as shown in FIG. 3, the controller 30 controls the valve v1 to be in an open state and forms the first flow channel fc1 from the blood bag bg to the first syringe 21 via the pipe 2, the valve v1, the joint j1, and the pipe 2. In addition, the controller 30 controls the piston pulling action of the first syringe 21 to suck the blood in the blood bag bg into the first syringe 21 through the first flow channel fc1. As a result, the controller 30 moves a predetermined amount of blood from the blood bag bg to the first syringe 21. Thereafter, the controller 30 controls the valve v1 to be in a closed state so as to cancel the first flow channel fc1. Step ST1 is thus finished.

In the capturing step of step ST2, the controller 30 controls the valves v2 and v7 to be in an open state as shown in FIG. 4. A 21st flow channel fc21 from the first syringe 21 to the filter 24 via the pipe 2, the joint j1, the pipe 2, the valve v2, the joint j2, the pipe 2, the joint j3, and the pipe 2 is formed by the open state of the valve v2. In addition, a 22nd flow channel fc22 from the filter 24 to the drain container 25 via the pipe 2, the joint j4, the pipe 2, and the valve v7 is formed by the open state of the valve v7. In this state, the controller 30 controls the piston pushing action of the first syringe 21, sends the blood in the first syringe 21 to the filter 24 from above via the 21st flow channel fc21 and sends the blood component having passed through the filter 24 to the drain container 25 from the 22nd flow channel fc22 below. The filter 24 captures white blood cells contained in the blood by sending the blood in the first syringe 21 from the joint j2 to the filter 24. Red blood cells and platelets contained in the blood pass through the filter 24 and move to the drain container 25. At this time, the filter 24 is soaked with blood components, and the red blood cells as well as the trapped white blood cells remain at the filter 24. Similarly, blood remains in the 21st flow channel fc21 between the first syringe 21 and the filter 24. Similarly, blood components such as red blood cells remain in the 22nd flow channel fc22 between the filter 24 and the drain container 25. Thereafter, the controller 30 controls the valves v2 and v7 to be in a closed state so as to cancel the 21st flow channel fc21 and the 22nd flow channel fc22. Step ST2 is thus finished.

In the blood returning step of step ST3, the controller 30 controls the valves v3 and v2 to be in an open state as shown in FIG. 5. A 31st flow channel fc31 from the second syringe 22 to the joint j2 via the pipe 2, the joint j5, the pipe 2, and the valve v3 is formed by the open state of the valve v3. In addition, a 32nd flow channel fc32 from the joint j2 to the first syringe 21 via the pipe 2, the valve v2, the joint j1, and the pipe 2 is formed by the open state of the valve v2. In this state, the controller 30 controls the piston pulling action of the first syringe 21 and the piston pushing action of the second syringe 22 so as to be in synchronization with each other, sends the wash liquid in the second syringe 22 to the joint j2 via the 31st flow channel fc31 and sends the wash liquid having passed through the joint j2 from the 32nd flow channel fc32 toward the first syringe 21. Note that by sending the wash liquid in the second syringe 22 from the joint j2 toward the first syringe 21, the blood remaining in the joint j2 is pushed back toward the first syringe 21. In this event, the controller 30 sends the wash liquid to at least a position closer to the first syringe 21 than the valve v2. In addition, the controller 30 may send the wash liquid to the first syringe 21. In any case, the blood remaining in the joint j2 and the pipe 2 between the valve v2 and the joint j2 is pushed back to the upstream side of the valve v2. Thereafter, the controller 30 controls the valves v3 and v2 to be in a closed state so as to cancel the 31st flow channel fc31 and the 32nd flow channel fc32. Step ST3 is thus finished. Note that only one of the piston pulling action of the first syringe 21 and the piston pushing action of the second syringe 22 may be controlled, and the other piston may be opened without applying a force. In this case, for example, the first syringe 21 is lifted by a positive pressure transmitted through the flow channel by the piston pushing action of the second syringe 22.

In the washing step of step ST4, the controller 30 controls the valves v3 and v7 to be in an open state as shown in FIG. 6. A fourth flow channel fc4 from the second syringe 22 to the drain container 25 via the pipe 2, the joint j5, the pipe 2, the valve v3, the joint j2, the pipe 2, the joint j3, the pipe 2, the filter 24, the pipe 2, the joint j4, the pipe 2, and the valve v7 is formed by the open states of the valves v3 and v7. In this state, the controller 30 controls the piston pushing action of the second syringe 22, sends the wash liquid in the second syringe 22 from the joint j2 to the filter 24 via the fourth flow channel fc4 and sends the wash liquid having passed through the filter 24 to the drain container 25. In this event, the red blood cells remaining in the filter 24 are washed away by the wash liquid and move to the drain container 25. This places the filter 24 in a washed state while the filter 24 keeps holding the white blood cells. The flow channel between the joint j2 and the drain container 25 is also placed in a washed state. Thereafter, the controller 30 controls the valves v3 and v7 to be in a closed state so as to cancel the fourth flow channel fc4. Step ST4 is thus finished. The above steps ST1 to ST4 may be referred to as liquid delivery operation. In addition, the following steps ST5 to ST6 may be referred to as collection operation.

In the collection step of step ST5, the controller 30 controls the valves v4 to v6, and v9 to be in the open state as shown in FIG. 7. A fifth flow channel fc5 from the second syringe 22 to the third syringe 23 via the pipe 2, the joint j5, the pipe 2, the valves v4 and v6, the joint j4, the pipe 2, the filter 24, the pipe 2, the joint j3, the pipe 2, the valves v5 and v9, the joint j6, and the pipe 2 is formed by the open states of the valves v4 to v6 and v9. In this state, the controller 30 controls piston pulling action of the third syringe 23 and piston pushing action of the second syringe 22 so as to be in synchronization with each other, sends the wash liquid in the second syringe 22 to the filter 24 from below via the fifth flow channel fc5 and sends the wash liquid having passed through the filter 24 to the third syringe 23 together with white blood cells from below. The wash liquid in the second syringe 22 is sent to the third syringe 23 through the filter 24 from below, so that the white blood cells captured by the filter 24 are collected to the third syringe 23 together with the wash liquid. Thereafter, the controller 30 controls the valves v4 to v6 and v9 to be in a closed state so as to cancel the fifth flow channel fc5. Step ST5 is thus finished. Note that only one of the piston pulling action of the third syringe 23 and the piston pushing action of the second syringe 22 may be controlled, and the other piston may be opened without applying a force.

In the extraction step of step ST6, the controller 30 controls the valves v8 and v10 to be in an open state as shown in FIG. 8. A 61st flow channel fc61 from the third syringe 23 to the spiral flow channel 26 via the pipe 2, the joint j6, the pipe 2, and the valve v8 is formed by the open state of the valve v8. Further, a 62nd flow channel fc62 from the spiral flow channel 26 to the drain container 25 via the pipe 2 is always formed. Further, a 63rd flow channel fc63 from the spiral flow channel 26 to the drain container 25 via the pipe 2, the joint j7, the pipe 2 and the valve v10 is formed by the open state of the valve v10. In this state, the controller 30 controls the piston pushing action of the third syringe 23, sends the wash liquid containing white blood cells in the third syringe 23 to the spiral flow channel 26 via the 61st flow channel fc61, and sends the wash liquid having passed through the spiral flow channel 26 to the drain container 25 individually via the 62nd flow channel fc62 and the 63rd flow channel fc63. Note that FIG. 8 shows initial operation of step ST6. At the initial stage of step ST6, a flow rate of the wash liquid passing through the spiral flow channel 26 is low, and thus, the CD34 positive cells are not extracted from the white blood cells contained in the wash liquid, and thus, the wash liquid is sent from the 63rd flow channel fc63 to the drain container 25.

Next, as shown in FIG. 9, the controller 30 controls the valve v10 to be in a closed state and controls the valve v11 to be in an open state. The closed state of the valve v10 cancels the 63rd flow channel fc63. On the other hand, a 64th flow channel fc64 from the spiral flow channel 26 to the collection container 27 via the pipe 2, the joint j7, the pipe 2, and the valve v11 is formed by the open state of the valve v11. In this state, the controller 30 continuously controls the piston pushing action of the third syringe 23, sends the wash liquid containing white blood cells in the third syringe 23 to the spiral flow channel 26 via the 61st flow channel fc61, sends the wash liquid having passed through the spiral flow channel 26 from the 62nd flow channel fc62 to the drain container 25, and sends the CD34 positive cells extracted by the spiral flow channel 26 from the 64th flow channel fc64 to the collection container 27. Note that FIG. 9 shows the operation of step ST6. In the operation of step ST6, a flow rate of the wash liquid passing through the spiral flow channel 26 is high, and thus, the CD34 positive cells are extracted from the white blood cells contained in the wash liquid, and thus, the CD34 positive cells are sent from the 64th flow channel fc64 to the collection container 27. Thereafter, the controller 30 controls the valves v8 and v11 to be in a closed state so as to cancel the 61st flow channel fc61 and the 64th flow channel fc64. Step ST6 is thus finished. The CD34 positive cells in the collection container 27 can be cultured by an expansion culture step.

As described above, according to one embodiment, the white blood cell collection apparatus 1 includes the first syringe 21 as the first container, the second syringe 22 as the second container, the filter 24, the joint j2 as the branching portion, and the controller 30. The first syringe 21 stores blood. The second syringe 22 stores the wash liquid. The blood sent from the first syringe 21 and the wash liquid sent from the second syringe 22 pass through the filter 24. The joint j2 communicates with each of the first syringe 21, the second syringe 22, and the filter 24. The controller 30 controls the liquid delivery operation through the joint j2. The liquid delivery operation includes the filter 24 capturing white blood cells contained in the blood by sending the blood in the first syringe 21 from the joint j2 to the filter 24, pushing back the blood remaining in the joint j2 toward the first syringe 21 by sending the wash liquid in the second syringe 22 from the joint j2 toward the first syringe 21 after the capturing, and washing the filter 24 by sending the wash liquid in the second syringe 22 from the joint j2 to the filter 24 after the pushing back. As described above, with the configuration in which the liquid delivery operation is controlled by the controller 30, a process of sending the fluid, a process of switching the flow channel, and a process requiring a skill of a user can be automated, so that stable quality can be secured regardless of the skill of the user. In addition, with the configuration in which the wash liquid in the second syringe 22 is sent from the joint j2 to the filter 24 after the blood remaining in the joint j2 is pushed back toward the first syringe 21, it is possible to suppress decrease in cleanliness of the wash liquid before washing the filter.

To be more specific, the white blood cell collection method in related art usually has no particular problem, but according to the study of the present inventor, in a case where the joint portion between the first pipe through which the blood flows and the second pipe through which the wash liquid flows is on the upstream side of the filter, there is a possibility that the blood flows into the wash liquid at the time of washing. Specifically, during washing, blood remaining in the first pipe and the joint portion may flow into the wash liquid flowing into the joint portion from the second pipe. In this case, the cleanliness of the wash liquid is lowered before washing the filter. Note that the cleanliness of the wash liquid is desirably maintained before washing the filter.

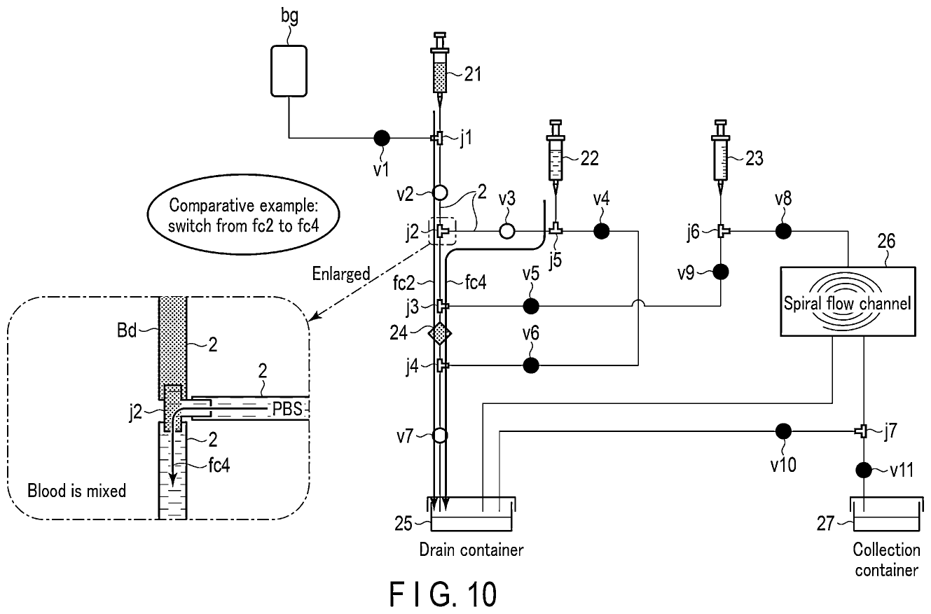

Specifically, in a comparative example, the pushing back step of step ST3 is not performed, and the capturing step of step ST2 is switched to the washing step of step ST4. In this comparative example, as shown in FIG. 10, after step ST2, blood remains in the second flow channel fc2 between the first syringe 21 and the drain container 25. If step ST4 is performed in this state, the blood is washed in the fourth flow channel fc4 from the second syringe 22 to the drain container 25 via the joint j2, but the blood Bd is mixed into the joint j2 from the pipe 2 on the first syringe 21 side. This lowers the cleanliness of the wash liquid (PBS) in the fourth flow channel fc4 downstream of the joint j4.

On the other hand, according to one embodiment,

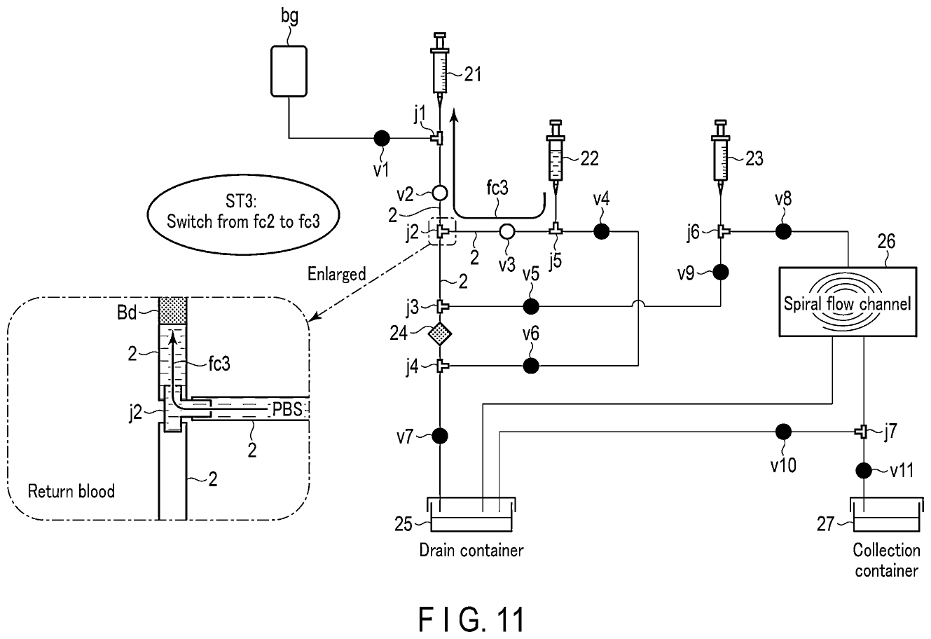

as shown in FIG. 11, the second flow channel fc2 is switched to the third flow channel fc3, and thus, the wash liquid in the third flow channel fc3 from the second syringe 22 to the first syringe 21 via the joint j2 pushes back the blood Bd remaining in the joint j2 of the second flow channel fc2. Thereafter, the flow channel is switched to the fourth flow channel fc4. Thus, as described above, it is possible to suppress decrease in cleanliness of the wash liquid before washing the filter.

In addition, according to one embodiment, the valve v2 controlled to be open and closed by the controller 30 may be provided between the joint j2 and the first syringe 21. In addition, the capturing may include controlling the valve v2 to be in an open state. The pushing back may include controlling the valve v2 to be in an open state and sending the wash liquid to at least a position closer to the first syringe 21 than the valve v2. The washing may include controlling the valve v2 to be in a closed state. In this case, in addition to the effects described above, with the configuration in which the valve v2 is closed after the blood is pushed back to a position closer to the first syringe 21 than the valve v2, it is possible to prevent part of the pushed back blood from being mixed into the wash liquid.

Also, according to one embodiment, the pushing back may include sending the wash liquid to the first syringe 21. In this case, in addition to the effects described above, it is not necessary to adjust a position where the blood is to be pushed back between the valve v2 and the first syringe 21, so that the liquid delivery operation can be easily controlled.

In addition, according to one embodiment, the branching portion may be the joint j2 that connects the pipe 2 of the flow channel communicating with the first syringe 21, the pipe 2 of the flow channel communicating with the second syringe 22, and the pipe 2 of the flow channel communicating with the filter 24. In this case, easy implementation can be achieved in addition to the effects described above.

In addition, according to one embodiment, a spiral flow channel 26 through which white blood cells are to be sent may be further provided. The controller 30 may further control collection operation different from the liquid delivery operation. The collection operation may include collecting white blood cells from the filter 24 after washing the filter 24 and sending the collected white blood cells to the spiral flow channel 26 so that the spiral flow channel 26 extracts CD34 positive cells from the white blood cells. In this case, in addition to the effects described above, CD34 positive cells can be collected from white blood cells contained in blood.

### Modification 1

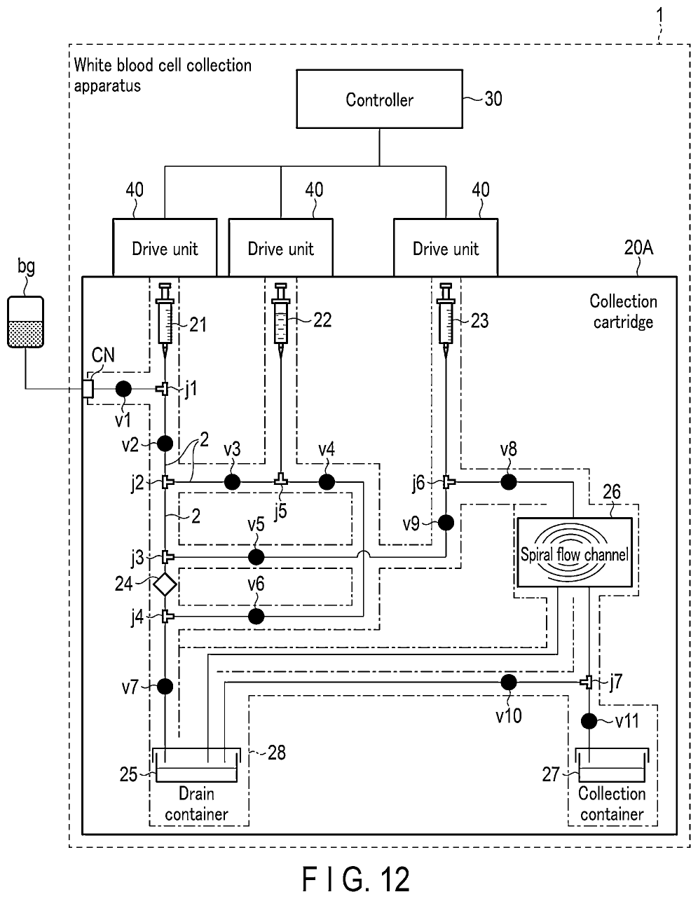

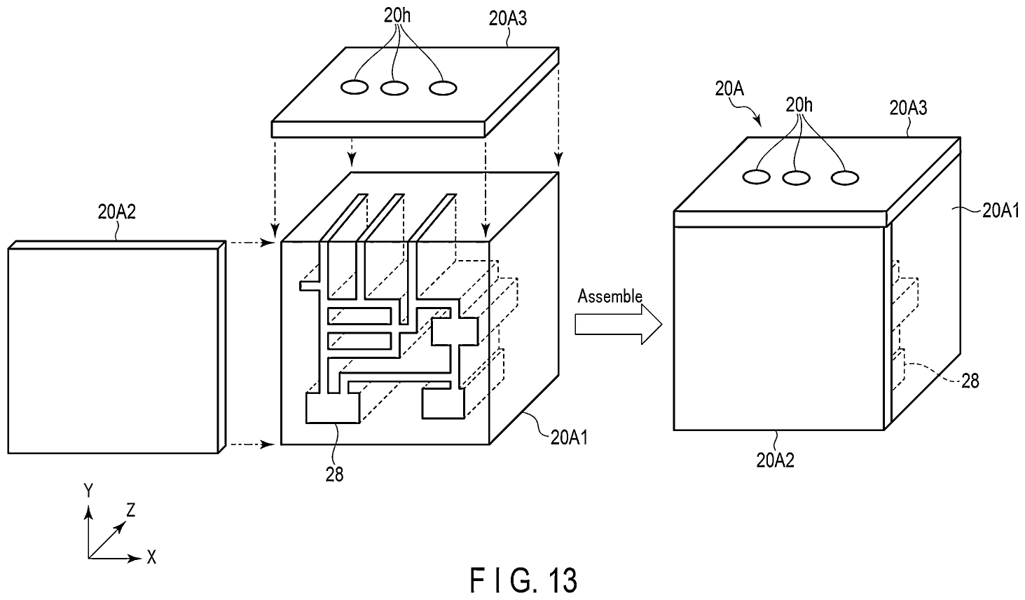

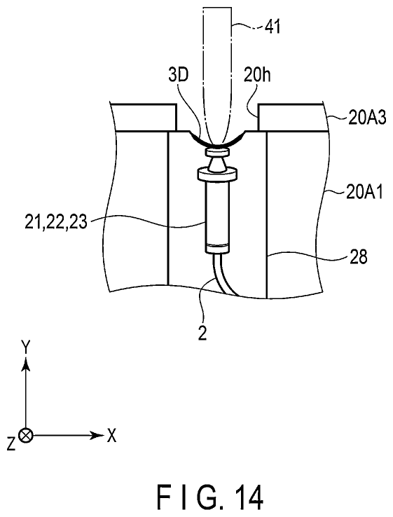

In one embodiment, the collection unit 20 in which various closed-system flow channels are switchably formed has been described, but the present invention is not limited thereto. For example, as shown in FIG. 12, the collection unit 20 may be implemented as a collection cartridge 20A. The collection cartridge 20A is detachably held with respect to a drive unit 40 that is controlled by the controller 30 and drives the first syringe 21, the second syringe 22, and the third syringe 23. The drive unit 40 includes, for example, a power source such as a motor and a pressing portion for transmitting a force by the power source to the first to third syringes. A bar-shaped member 41 described later is an example of the pressing portion. In addition, a compressor may be used as a power source, and a piston of an air cylinder connected to the compressor may be used as the pressing portion. The collection cartridge 20A includes a closed-system flow channel and a closed-system container that accommodates the flow channel. Similarly to the above, the closed-system flow channel includes a first syringe 21, a second syringe 22, a third syringe 23, a filter 24, a drain container 25, a spiral flow channel 26, a collection container 27, a plurality of pipes 2, joints j1 to j7, valves v1 to v11, and a connector CN. The closed-system flow channel has a first closed structure and is held by a holder 28 formed along the flow channel. The holder 28 may be a groove or a guide plate. The closed-system container has a second closed structure disposed outside the flow channel of the first closed structure so that the liquid does not leak to the outside of the collection cartridge 20A. In other words, the collection cartridge 20A has a double closed structure including the first closed structure and the second closed structure. As shown in FIG. 13, for example, the container of the collection cartridge 20A includes a container main body 20A1 in which a recess as the holder 28 is formed, a first lid 20A2 attached to a side surface of the container main body 20A1 so as to cover the recess, and a second lid 20A3 attached to an upper surface of the container main body 20A1. In other words, the collection cartridge 20A is assembled, for example, by attaching the first lid 20A2 to the side surface of the container main body 20A1 and attaching the second lid 20A3 to the upper surface of the container main body 20A1. The second lid 20A3 has holes 20h formed at positions corresponding to the first syringe 21, the second syringe 22, and the third syringe 23. The container main body 20A1, the first lid 20A2, and the second lid 20A3 are hard and made of, for example, resin or plastic. The resin material is, for example, polycarbonate (PC) or polystyrene (PS). At least one of the container main body 20A1 or the first lid 20A2 is preferably light transmitting property. For example, at least one of the container main body 20A1 or the first lid 20A2 preferably has visible light transmitting property from the viewpoint that the white blood cell collecting process can be easily observed. FIG. 13 shows an appearance in a case where the container main body 20A1 is transparent and the first lid 20A2 is opaque. The recess as the holder 28 has a depth in a Z direction orthogonal to the closed-system flow channel and is formed of a hole and a groove that accommodate the closed-system flow channel. A Y direction is a direction connecting the first syringe 21 and the drain container 25, and is, for example, a vertical direction. An X direction is a direction orthogonal to the Y direction and the Z direction. As shown in FIG. 14, the hole 20h of the second lid 20A3 is located on the recess as the holder 28 and includes a diaphragm 3D at a position covering the recess. In other words, the second lid 20A3 includes the diaphragms 3D at positions covering the first syringe 21, the second syringe 22, and the third syringe 23 in the flow channel. The diaphragm 3D is an example of an energy transmission portion that receives energy for sending a fluid in the closed-system flow channel from the outside of the container. The diaphragm 3D is located at a boundary separating the inside and the outside of the container. The bar-shaped member 41 of the drive unit 40 penetrates the diaphragm 3D and is attached to each piston of the first syringe 21, the second syringe 22, and the third syringe 23. However, the bar-shaped member 41 of the drive unit 40 performs only the pushing action for the second syringe 22, so that it is not always necessary to attach the second syringe 22 by penetrating the diaphragm 3D.

In addition to the above-described configuration, the controller 30 controls the drive unit 40 to drive the first syringe 21 and the second syringe 22 in a state where the collection cartridge 20A is attached to the drive unit 40, thereby controlling the liquid delivery operation. In other words, in the liquid delivery operation, the capturing includes controlling the drive unit 40 to discharge the blood in the first syringe 21. In the liquid delivery operation, the pushing back includes controlling the drive unit 40 to cause the blood to flow into the first syringe 21 and cause the wash liquid in the second syringe 22 to flow toward the joint j2. In the liquid delivery operation, the washing includes controlling the drive unit 40 to discharge the wash liquid in the second syringe 22. In the liquid delivery operation of pushing back from the second syringe 22 toward the first syringe 21, similarly to the above, the piston pulling action of the first syringe 21 and the piston pushing action of the second syringe 22 may be controlled in synchronization with each other. Alternatively, only one of the piston pulling action of the first syringe 21 and the piston pushing action of the second syringe 22 may be controlled, and the other piston may be opened without applying a force.

Similarly, the controller 30 controls the drive unit 40 to drive the second syringe 22 and the third syringe 23 in a state where the collection cartridge 20A is attached to the drive unit 40, thereby controlling the collection operation. In other words, in the collection operation, the collection includes causing the drive unit 40 to discharge the wash liquid in the second syringe 22 and causing the third syringe 23 to suck the wash liquid containing white blood cells. In the collection operation, the extracting includes controlling the drive unit 40 to discharge the wash liquid containing white blood cells in the third syringe 23 toward the spiral flow channel 26. In the collection operation of collecting white blood cells by causing the wash liquid to flow from the second syringe 22 toward the third syringe 23, the piston pulling action of the third syringe 23 and the piston pushing action of the second syringe 22 may be controlled in synchronization with each other as described above. Alternatively, only one of the piston pulling action of the third syringe 23 and the piston pushing action of the second syringe 22 may be controlled, and the other piston may be opened without applying a force.

The drive unit 40 is controlled by the controller 30 and drives each of the first syringe 21 that stores blood, the second syringe 22 that stores the wash liquid, and the third syringe 23 that collects white blood cells so as to perform discharge and suction. Specifically, the drive unit 40 drives each piston via the bar-shaped member 41 so that each piston of the first syringe 21, the second syringe 22, and the third syringe 23 performs the pushing action and the pulling action. Out of the two syringes that become a start point and an end point of the flow of the liquid, the drive unit 40 may be driven by the control of the controller 30 in synchronization with the piston pulling action of the syringe that becomes the end point and the piston pushing action that becomes the start point. Alternatively, out of the two syringes, only one of the piston pulling action of the syringe that becomes the end point and the piston pushing action of the syringe that becomes the start point may be driven, and the other piston may be opened without applying a force.

Other configurations are similar to those of the embodiment.

According to Modification 1 as described above, the drive unit 40 that is controlled by the controller 30 and drives the first syringe 21 and the second syringe 22 is further provided. The first syringe 21, the second syringe 22, the filter 24, and the joint j2 are accommodated in the collection cartridge 20A detachable from the drive unit 40. The controller 30 controls the drive unit 40 to drive the first syringe 21 and the second syringe 22 in a state where the collection cartridge 20A is attached to the drive unit 40, thereby controlling the liquid delivery operation. As described above, with the configuration using the detachable collection cartridge 20A as the collection unit 20, the closed-system flow channel after collection can be disposable every time white blood cells (CD34 positive cells) are collected from the blood of the donor, in addition to the effects described above. Thus, by replacing the collection cartridge 20A for each blood of the donor, the step of washing the entire closed-system flow channel can be omitted during the white blood cell collecting process for each donor, so that a collection amount of white blood cells per unit time can be increased. That is, an improved throughput for the white blood cell collecting process can be realized.

In addition, according to Modification 1, the collection cartridge 20A may include the closed-system flow channel configured to include the first syringe 21, the second syringe 22, the filter 24, and the joint j2, and the closed-system container that accommodates the flow channel. In this case, even if the closed-system flow channel is damaged and a fluid leaks, the closed-system container does not allow the leaked fluid to flow out to the outside. Thus, in addition to the effects discussed above, safety can be secured without necessitating a facility investment associated with the risk of liquid leakage. To be more specific, according to Modification 1, it is possible to ensure safety locally in the collection cartridge 20A, not in a global facility such as a safety cabinet or a laboratory.

According to Modification 1, the first container is the first syringe 21 that stores blood and is driven by the drive unit 40. The second container is the second syringe 22 that stores the wash liquid and is driven by the drive unit 40. In the liquid delivery operation, the capturing includes controlling the drive unit 40 to discharge the blood in the first syringe 21. The pushing back includes controlling the drive unit 40 to cause the blood to flow into the first syringe 21 and cause the wash liquid in the second syringe 22 to flow toward the joint j2. The washing includes controlling the drive unit 40 to discharge the wash liquid in the second syringe 22. Thus, as described above, it is possible to implement quantitative and accurate liquid delivery by the syringe.

Further, according to Modification 1, the diaphragm 3D is provided so as to be positioned at a boundary separating the inside and outside of the closed-system container. As a result, in addition to the effects described above, in a case where a liquid delivery amount is relatively small, if the fluid leaks from the flow channel, the leaked fluid can be prevented from flowing out to the outside of the container by the simple configuration of the diaphragm having a small displacement amount.

### Modification 2

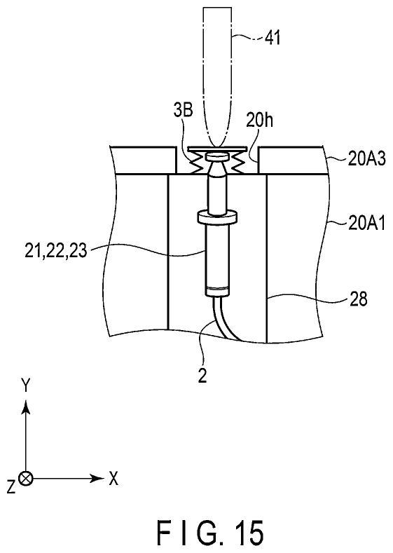

In Modification 1 of the embodiment, the diaphragm 3D is provided so as to be positioned at the boundary separating the inside and the outside of the closed-system container, but the present invention is not limited thereto. For example, as shown in FIG. 15, the collection cartridge 20A may include a bellows 3B instead of the diaphragm 3D. According to Modification 2, in addition to the effects of Modification 1, in a case where the liquid delivery amount is relatively large, if the fluid leaks from the flow channel, the leaked fluid can be prevented from flowing out of the container by the simple configuration of the bellows having a large displacement amount.

### Modification 3

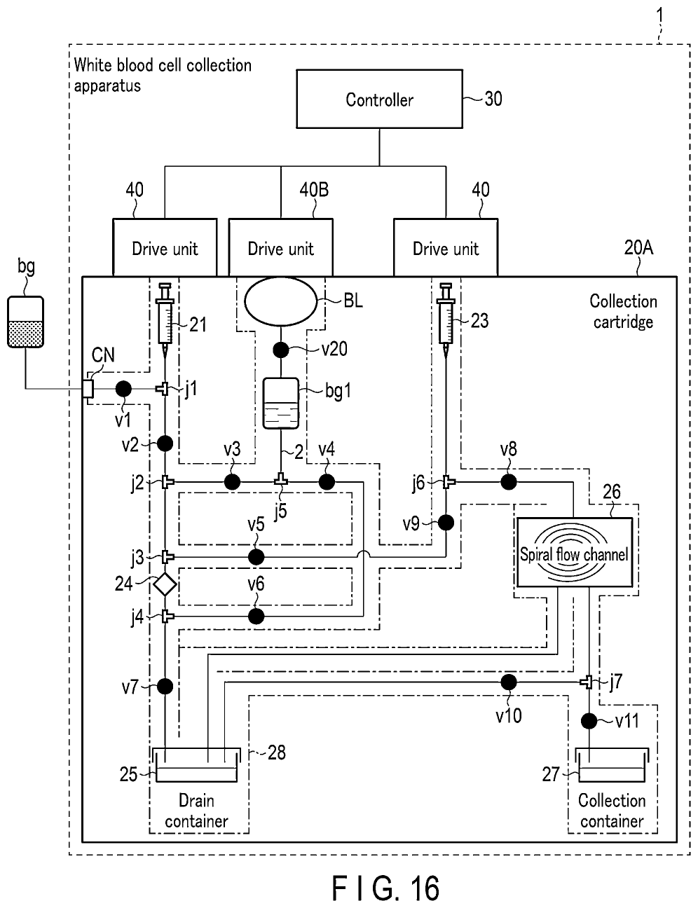

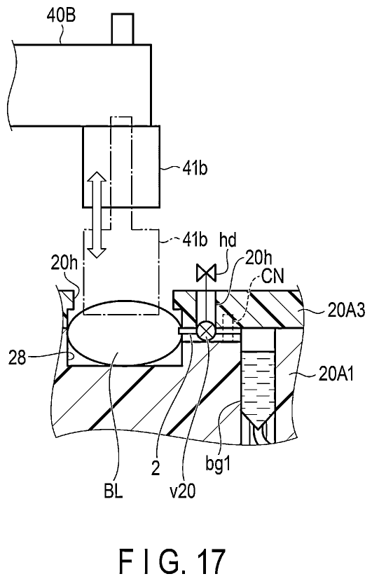

In Modification 1 and Modification 2 of the embodiment, the drive unit 40 drives the second syringe 22, but the present invention is not limited thereto. The second syringe 22 performs only discharge out of discharge and suction, and thus, can be modified to have a configuration dedicated to discharge such as a balloon. For example, as shown in FIGS. 16 and 17, the collection cartridge 20A may include, instead of the second syringe 22, a bag bg1 as a storage unit that stores a wash liquid, and a balloon BL which is connected to the bag bg1, holds gas, and is driven by the drive unit 40B. The bag bg1 may be a flexible container. The flexible container may be a plastic or vinyl container. Here, a pipe 2 at an upper end of the bag bg1 is connected to the balloon BL via the connector CN and the valve v20. If a detachable handle hd is attached, the valve v20 can be open and closed according to operation of the handle hd. The balloon BL is pressed by a columnar member 41b of the drive unit 40B to introduce the stored gas to the upper end of the bag bg1 via the valve v20, the connector CN, and the pipe 2. The bag bg1 discharges the wash liquid pressed by the introduced gas from the pipe 2 at a lower end toward the joint j5. The configuration including the bag bg1 and the balloon BL is another example of the second container that sends the wash liquid.

In the above-described configuration, the controller 30 controls the drive unit 40 to drive the first syringe 21 and the balloon BL in a state where the collection cartridge 20A is attached to the drive unit 40, thereby controlling the liquid delivery operation. In other words, in the liquid delivery operation, the capturing includes controlling the drive unit 40 to discharge the blood in the first syringe 21. In the liquid delivery operation, the pushing back includes controlling the drive unit 40 to cause blood to flow into the first syringe 21 and controlling the drive unit 40B to discharge the gas in the balloon BL to the bag bg1 to discharge the wash liquid in the bag bg1 toward the joint j2. In the liquid delivery operation, the washing includes controlling the drive unit 40B to discharge the gas in the balloon BL to the bag bg1 and discharge the wash liquid in the bag bg1.

The drive unit 40B is controlled by the controller 30 and drives the balloon BL connected to the bag bg1 that stores the wash liquid so as to discharge the wash liquid from the bag bg1. Specifically, the drive unit 40B drives the balloon BL via the columnar member 41b so as to press the balloon BL.

Other configurations are similar to those of Modification 1 and Modification 2 of the embodiment.

According to Modification 3 as described above, the first container is the first syringe 21 that stores blood and is driven by the drive unit 40. The second container includes the bag bg1 that stores the wash liquid, and the balloon BL which is connected to the bag bg1, holds gas, and is driven by the drive unit 40B. In the liquid delivery operation, the capturing includes controlling the drive unit 40 to discharge the blood in the first syringe 21. The pushing back includes controlling the drive unit 40 to cause blood to flow into the first syringe 21 and controlling the drive unit 40B to discharge the gas in the balloon BL to the bag bg1 to discharge the wash liquid in the bag bg1 toward the joint j2. In the liquid delivery operation, the washing includes controlling the drive unit 40B to discharge the gas in the balloon BL to the bag bg1 and discharge the wash liquid in the bag bg1. Thus, in addition to the effects described above, it is possible to implement simple liquid delivery that does not require quantification by the balloon.

### Modification 4

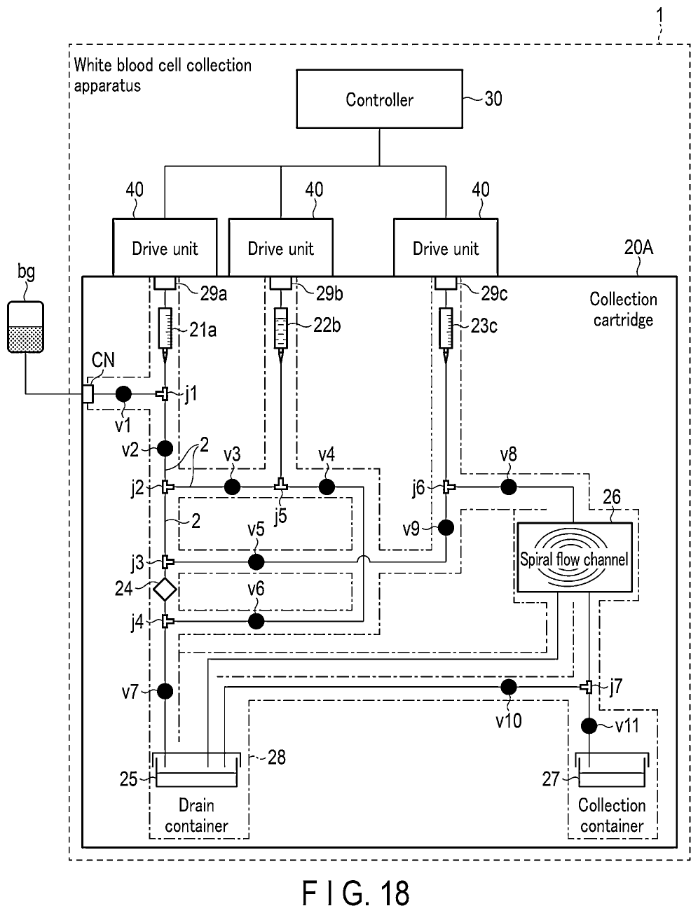

In one embodiment and Modifications 1 to 3 thereof, the syringe or the balloon is used as the container, and a mechanism that presses the syringe or the balloon is used as the drive unit, but the present invention is not limited thereto. For example, as shown in FIG. 18, the first to third containers may include storage containers 21a, 22b, and 23c, and connection portions 29a, 29b, and 29c that communicate with the storage containers and aseptically communicate with the outside of the collection cartridge 20A via an air filter. In this case, a pumping mechanism that generates a positive and/or negative gas pressure can be used as the drive unit 40. Each pumping mechanism may be constituted by a syringe pump or by a compressor and an air regulator, etc., and any suitable gas-producing mechanism may be employed as the pumping mechanism. The drive unit 40 is connected to the connection portions 29a, 29b, and 29c, and drives operation of applying a positive pressure to discharge the liquid in the storage containers 21a, 22b, and 23c or drives operation of applying a negative pressure to suck the liquid into the storage containers 21a and 23c. The storage container 21a is an example of a first storage unit that stores blood. The storage container 22b is an example of a second storage unit that stores the wash liquid. The connection portion 29a is an example of a first connection portion connected to the pumping mechanism. The connection portion 29b is an example of a second connection portion connected to the pumping mechanism.

In order to execute the operation in steps ST1 to ST6 according to the configuration of the present modification, the operation of applying a positive pressure to the storage containers 21a, 22b, and 23c, respectively, may be executed instead of the pushing action of the first to third syringes 21, 22, and 23 in the above-described embodiment, and the operation of applying a negative pressure to the storage containers 21a and 23c, respectively, may be executed instead of the pulling action of the first and third syringes 21 and 23. For example, in step ST2, the capturing includes controlling the drive unit 40 to apply a positive pressure to the storage container 21a to discharge blood from the storage container 21a. In step ST3, the pushing back includes controlling the drive unit 40 to apply a negative pressure to the storage container 21a and a positive pressure to the storage container 22b to generate a flow in a direction from the storage container 22b toward the storage container 21a. In step ST4, the washing includes controlling the drive unit 40 to discharge the wash liquid in the storage container 22b.

However, in executing the blood returning step ST3 according to the configuration of the present modification, it is not always necessary to execute both the operation of applying a negative pressure to the storage container 21a and the operation of applying a positive pressure to the storage container 22b, and it is sufficient to execute only one of the operation. In other words, in the operation of step ST3, the flow in the direction from the storage container 22b to the storage container 21a may be generated by at least one of the operation of applying a negative pressure to the storage container 21a or the operation of applying a positive pressure to the storage container 22b.

Note that in FIG. 18, a mode in which the drive units 40 are simultaneously connected to the connection portions 29a, 29b, and 29c is shown, but the present invention is not limited thereto. For example, in each step of ST1 to ST6, the drive unit 40 may be configured to move such that the drive unit 40 is connected to one of the connection portions 29a, 29b, and 29c that requires application of a gas pressure.

According to Modification 4 as described above, a movable portion mechanism in the collection cartridge 20A can be reduced, so that the collection cartridge 20A can be manufactured at lower cost.

### Modification 5

In Modifications 1 to 4 of one embodiment, the holder 28 of the collection cartridge 20A has a layout capable of accommodating only one type of closed-system flow channel, but the present invention is not limited thereto. For example, the collection cartridge 20A may have a layout capable of accommodating two types of closed-system flow channels. In this case, the container main body 20A1 of the collection cartridge 20A includes the holder 28 having a layout including a recess to be used in both of the two flow channels, a recess to be used only in one flow channel, and a recess to be used only in the other flow channel. Similarly, the collection cartridge 20A may have a layout capable of accommodating three or more types of closed-system flow channels. According to Modification 5, in addition to the effects of Modifications 1 to 4 of one embodiment, versatility of the collection cartridge 20A can be improved.

### Modification 6

In Modifications 1 to 5 of one embodiment, the collection cartridge 20A has a double closed structure such as the closed-system flow channel and the closed-system container for accommodating the flow channel, but the present invention is not limited thereto. For example, the collection cartridge 20A may have a triple closed structure including a container that further accommodates the double closed structure. Alternatively, the collection cartridge 20A is not limited to the triple closed structure and may have a multiple closed structure of four or more. According to Modification 6 as described above, in addition to the effects of Modifications 1 to 5 of one embodiment, it can be expected that the closed-system flow channel can be protected from impact when the collection cartridge 20A is dropped, so that it is possible to further improve safety.

According to at least one embodiment described above, it is possible to suppress decrease in cleanliness of the wash liquid before washing the filter.

The term “processor” or the like used herein refers to, for example, a central processing unit (CPU) or a graphics processing unit (GPU), or various types of circuitry which may be an application-specific integrated circuit (ASIC), a programmable logic device (such as a simple programmable logic device (SPLD), a complex programmable logic device (CPLD), or a field programmable gate array (FPGA)), and so on. If, for example, the processor is a CPU, the processor reads and executes a program or programs stored in storage circuitry (not shown in the figures) to realize the intended functions. If, for example, the processor is an ASIC, the functions are directly incorporated into circuitry of the processor in the form of a logic circuit, instead of corresponding programs being stored in storage circuitry. Each processor in the embodiments, etc. is not limited to a single circuit-type processor, and multiple independent circuits may be combined as one processor to realize the functions. Furthermore, a plurality of components in FIGS. 1, 12, and 16 may be integrated into one processor to implement the functions.

Regarding the above embodiment, the following supplementary notes are disclosed as one aspect and selective features of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A white blood cell collection apparatus comprising:
a first container that stores blood;
a second container that stores a wash liquid;
a filter through which the blood sent from the first container and the wash liquid sent from the second container pass;
a branching portion communicating with each of the first container, the second container, and the filter; and
a controller configured to control liquid delivery operation through the branching portion,
wherein the liquid delivery operation includes:
sending blood in the first container from the branching portion to the filter to cause the filter to capture white blood cells contained in the blood;
pushing back the blood remaining in the branching portion toward the first container by sending the wash liquid in the second container from the branching portion toward the first container after the capturing; and
washing the filter by sending the wash liquid in the second container from the branching portion to the filter after the pushing back.

2. The white blood cell collection apparatus according to claim 1, further comprising:
a valve controlled to be open and closed by the controller, the valve being provided between the branching portion and the first container,
wherein
the controller controls the valve to be in an open state in the capturing,
the controller controls the valve to be in an open state by pushing back, thereby sending the wash liquid to at least a position closer to the first container than the valve, and
the controller controls the valve to be in a closed state in the washing.

3. The white blood cell collection apparatus according to claim 2,
wherein the liquid delivery operation includes sending the wash liquid to the first container by the pushing back.

4. The white blood cell collection apparatus according to claim 1,
wherein the branching portion is a joint that connects a pipe of a flow channel communicating with the first container, a pipe of a flow channel communicating with the second container, and a pipe of a flow channel communicating with the filter.

5. The white blood cell collection apparatus according to claim 1, further comprising:
a drive unit that is controlled by the controller and drives the first container and the second container, wherein
the first container, the second container, the filter, and the branching portion are accommodated in a cartridge detachable from the drive unit, and
the controller controls the liquid delivery operation by controlling the drive unit to drive the first container and the second container in a state where the cartridge is attached to the drive unit.

6. The white blood cell collection apparatus according to claim 5, wherein the cartridge includes a closed-system flow channel including the first container, the second container, the filter, and the branching portion, and a closed-system container that accommodates the flow channel.

7. The white blood cell collection apparatus according to claim 5, wherein
the first container is a first syringe that stores the blood and is driven by the drive unit, the second container is a second syringe that stores the wash liquid and is driven by the drive unit,
the controller controls the drive unit to discharge the blood in the first syringe in the capturing,
the controller controls the drive unit to cause blood to flow into the first syringe and cause the wash liquid in the second syringe to flow toward the branching portion in the pushing back, and
the controller controls the drive unit to discharge the wash liquid in the second syringe in the washing.

8. The white blood cell collection apparatus according to claim 5, wherein
the first container is a first syringe that stores the blood and is driven by the drive unit,
the second container includes a storage unit that stores the wash liquid, and a balloon which is connected to the storage unit, holds gas, and is driven by the drive unit,
the controller controls the drive unit to discharge the blood in the first syringe in the capturing,
the controller controls the drive unit to cause blood to flow into the first syringe and cause the gas in the balloon to be discharged to the storage unit to discharge the wash liquid in the storage unit in the pushing back, and
the controller controls the drive unit to discharge the gas in the balloon to the storage unit to discharge the wash liquid in the storage unit in the washing.

9. The white blood cell collection apparatus according to claim 5, wherein
the drive unit is a pumping mechanism that generates a positive and/or negative gas pressure,
the first container includes a first storage unit that stores the blood and a first connection portion connected to the pumping mechanism, and
the second container includes a second storage unit that stores the wash liquid and a second connection portion connected to the pumping mechanism,
the controller controls the drive unit to apply a positive pressure to the first storage unit to discharge blood from the first storage unit in the capturing,
the controller controls the drive unit to generate a flow in a direction from the second storage unit to the first storage unit in the pushing back, and
the controller controls the drive unit to discharge the wash liquid in the second storage unit in the washing.

10. The white blood cell collection apparatus according to claim 1, further comprising:
a spiral flow channel through which the white blood cells are to be sent, wherein
the controller further controls collection operation different from the liquid delivery operation, and
the collection operation includes:
collecting white blood cells from the filter after washing the filter; and
sending the collected white blood cells to the spiral flow channel so that the spiral flow channel extracts CD34 positive cells from the white blood cells.

11. A white blood cell collection method to be executed by a white blood cell collection apparatus comprising:
a first container that stores blood;
a second container that stores a wash liquid;
a filter through which the blood sent from the first container and the wash liquid sent from the second container pass; and
a branching portion communicating with each of the first container, the second container, and the filter,
the white blood cell collection method comprising:
sending the blood in the first container from the branching portion to the filter to cause the filter to capture white blood cells contained in the blood;
pushing back the blood remaining in the branching portion toward the first container by sending the wash liquid in the second container from the branching portion toward the first container after the capturing; and
washing the filter by sending the wash liquid in the second container from the branching portion to the filter after the pushing back.

12. The white blood cell collection method according to claim 11, wherein
the white blood cell collection apparatus includes a valve controlled to be open and closed between the branching portion and the first container,
the capturing includes controlling the valve to be in an open state,
the pushing back includes controlling the valve to be in an open state and sending the wash liquid to at least a position closer to the first container than the valve, and
the washing includes controlling the valve to be in a closed state.

13. The white blood cell collection method according to claim 12,
wherein the pushing back includes sending the wash liquid to the first container.

14. The white blood cell collection method according to claim 11,
wherein the branching portion is a joint that connects a pipe of a flow channel communicating with the first container, a pipe of a flow channel communicating with the second container, and a pipe of a flow channel communicating with the filter.

15. The white blood cell collection method according to claim 11, wherein
the white blood cell collection apparatus further includes a drive unit that drives the first container and the second container,
the first container, the second container, the filter, and the branching portion are accommodated in a cartridge detachable from the drive unit, and
the white blood cell collection apparatus executes the white blood cell collection method by controlling the drive unit to drive the first container and the second container in a state where the cartridge is attached to the drive unit.

16. The white blood cell collection method according to claim 15, wherein the cartridge includes a closed-system flow channel including the first container, the second container, the filter, and the branching portion, and a closed-system container that accommodates the flow channel.

17. The white blood cell collection method according to claim 15, wherein
the first container is a first syringe that stores the blood and is driven by the drive unit,
the second container is a second syringe that stores the wash liquid and is driven by the drive unit,
the capturing includes controlling the drive unit to discharge the blood in the first syringe,
the pushing back includes controlling the drive unit to cause blood to flow into the first syringe and cause the wash liquid in the second syringe to flow toward the branching portion, and
the washing includes controlling the drive unit to discharge the wash liquid in the second syringe.

18. The white blood cell collection method according to claim 15, wherein
the first container is a first syringe that stores the blood and is driven by the drive unit,
the second container includes a storage unit that stores the wash liquid, and a balloon which is connected to the storage unit, holds gas, and is driven by the drive unit,
the capturing includes controlling the drive unit to discharge the blood in the first syringe,
the pushing back includes controlling the drive unit to cause blood to flow into the first syringe and cause the gas in the balloon to be discharged to the storage unit to discharge the wash liquid in the storage unit, and
the washing includes controlling the drive unit to discharge the gas in the balloon to the storage unit to discharge the wash liquid in the storage unit.

19. The white blood cell collection method according to claim 15, wherein
the drive unit is a pumping mechanism that generates a positive and/or negative gas pressure,
the first container includes a first storage unit that stores the blood and a first connection portion connected to the pumping mechanism,
the second container includes a second storage unit that stores the wash liquid and a second connection portion connected to the pumping mechanism,
the capturing includes controlling the drive unit to apply a positive pressure to the first storage unit to discharge blood from the first storage unit,
the pushing back includes controlling the drive unit to generate a flow in a direction from the second storage unit toward the first storage unit, and
the washing includes controlling the drive unit to discharge the wash liquid in the second storage unit.

20. The white blood cell collection method according to claim 11, wherein
the white blood cell collection apparatus further includes a spiral flow channel through which the white blood cells are to be sent, and
the white blood cell collection method includes:
collecting white blood cells from the filter after washing the filter; and
sending the collected white blood cells to the spiral flow channel so that the spiral flow channel extracts CD34 positive cells from the white blood cells.
